(19) United States
(12) Plant Patent Application Publication
(10) Pub. No.: US 2006/0010549 P1
(43) Pub. Date: Jan. 12, 2006

(54) Mushroom variety named 'spoppo'

(75) Inventor: Johannes Jacobus Petrus Baars, Maasbree (NL)

Correspondence Address: THE WEBB LAW FIRM, P.C., 700 KOPPERS BUILDING, 436 SEVENTH AVENUE, PITTSBURGH, PA, 15219, US

(73) Assignee: Praktijkonderzoek Plant & Omgeving B.V., Wageningen (NL)

(21) Appl. No.: 11/176,960

(22) Filed: Jul. 7, 2005

(30) Foreign Application Priority Data
Jul. 7, 2004 (QZ) 2004/1334

(51) Int. Cl. A01H 15/00

(52) U.S. Cl. PLT/394

(57) ABSTRACT

‘Spoppo’ is a new variety of oyster mushroom that produces no spores and has well-developed fleshy caps.

### CROSS REFERENCE TO RELATED APPLICATION

The present application claims priority to European Community Plant Variety Rights Application No. 2004/1334, filed Jul. 7, 2004, which is herein incorporated by reference.

### BOTANICAL CLASSIFICATION

Pleurotus ostreatus

### VARIETAL DENOMINATION

‘Spoppo’

### BACKGROUND OF THE INVENTION

The present invention comprises a new and distinct cultivar of mushroom of the species Pleurotus ostreatus known by the varietal name ‘Spoppo’. The new variety was discovered in Horst-America. The purpose of the breeding program was to develop a sporeless strain of oyster mushroom. The new variety has been trial and field tested and has been found to retain its distinctive characteristics and remain true to type through successive propagations.

### DESCRIPTION OF THE DRAWINGS

The accompanying photographic drawings illustrate the new variety, with the color being as nearly true as is possible with color illustrations of this type:

FIG. 1 shows a plurality of ‘Spoppo’ mushrooms emerging from their growth medium;

FIG. 2 shows a comparison between ‘Spoppo’ on the right and HK35 on the left;

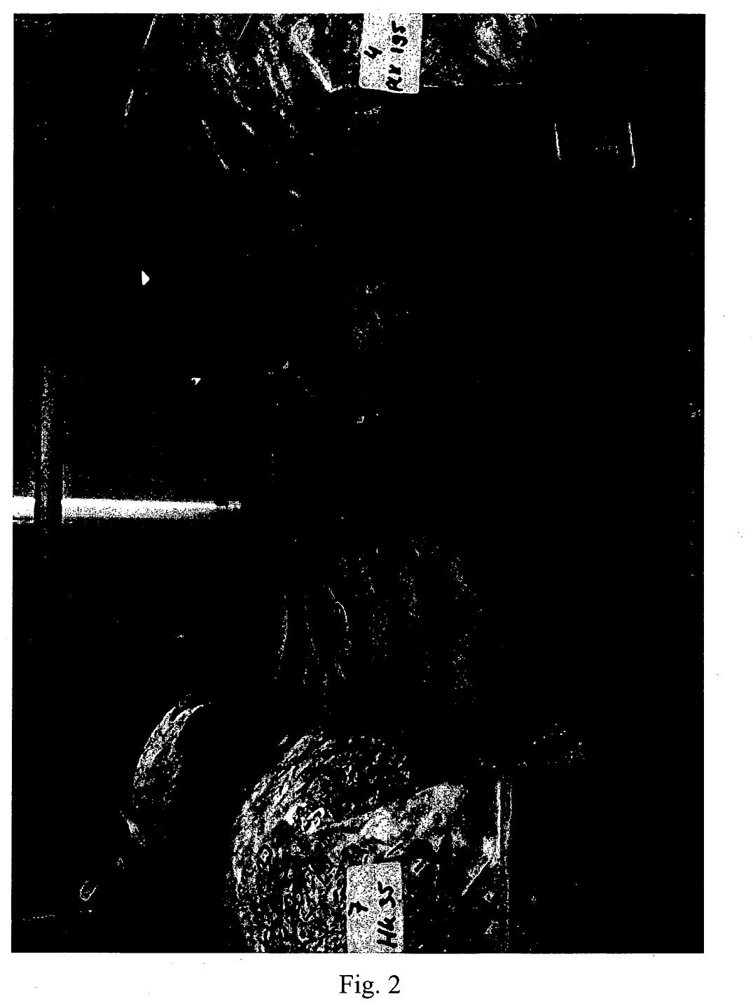

FIG. 3 is an upper side cap comparison between ‘Spoppo’ on the right and Mushroom #3 on the left;

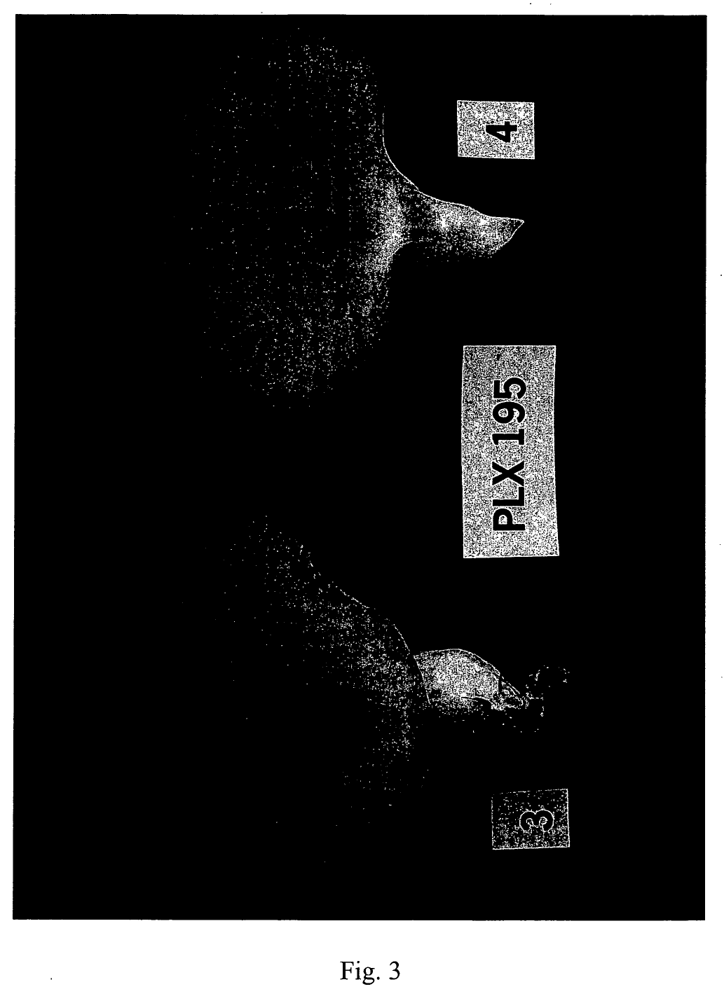

FIG. 4 is a lamellae comparison between ‘Spoppo’ on the right and Mushroom #3 on the left;

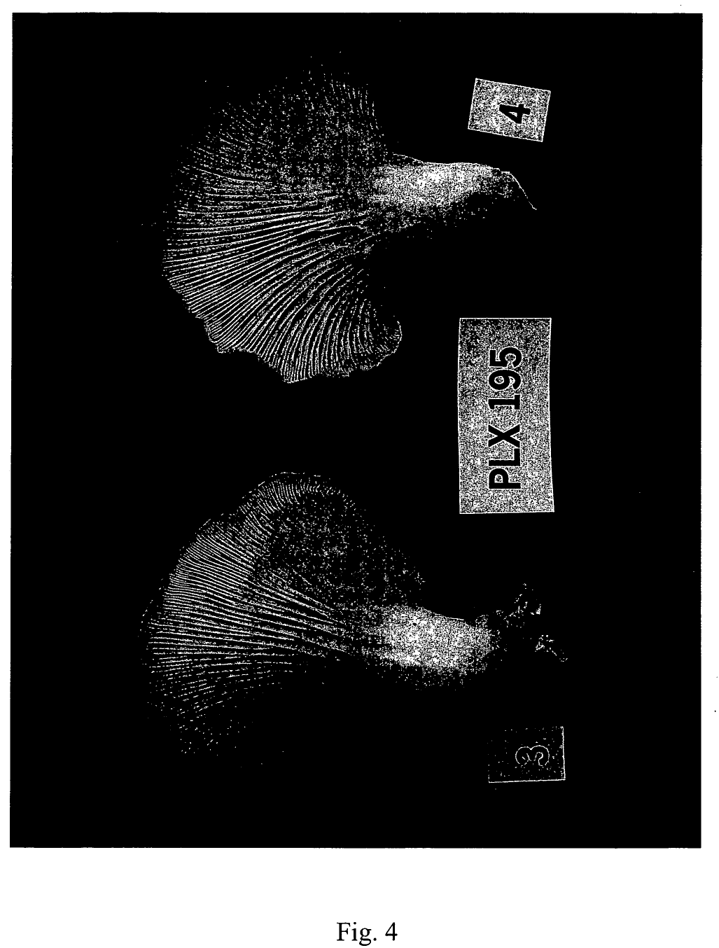

FIG. 5 indicates where the color of the upper side of the ‘Spoppo’ mushrooms was measured at location 2; and

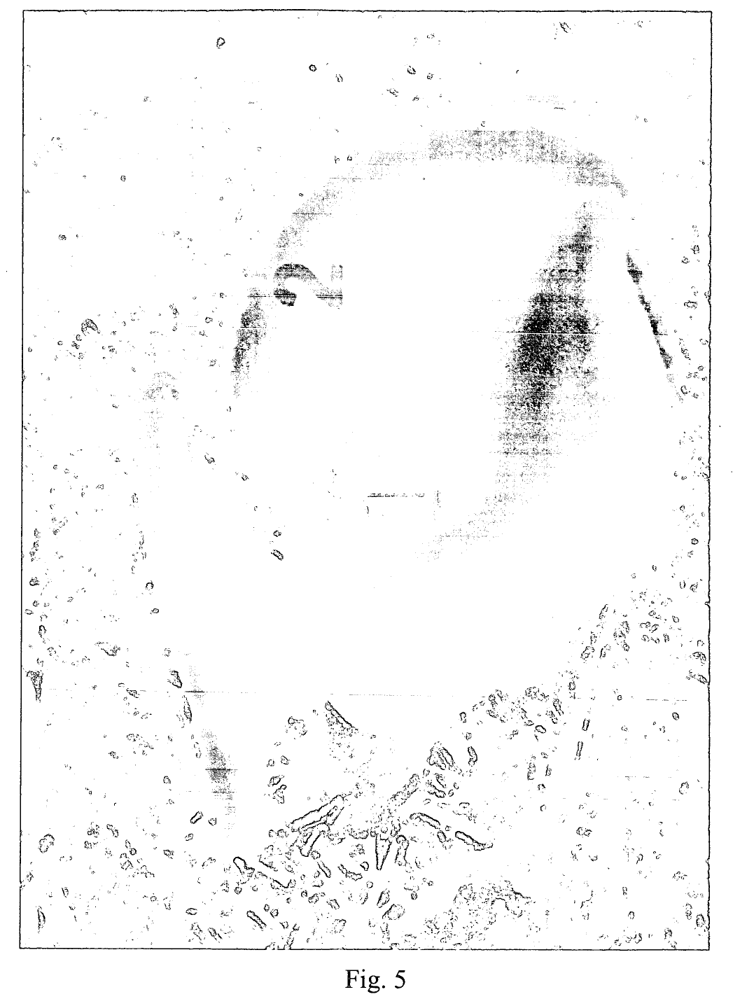

FIG. 6 indicates where the dimensions of ‘Spoppo’ were measured.

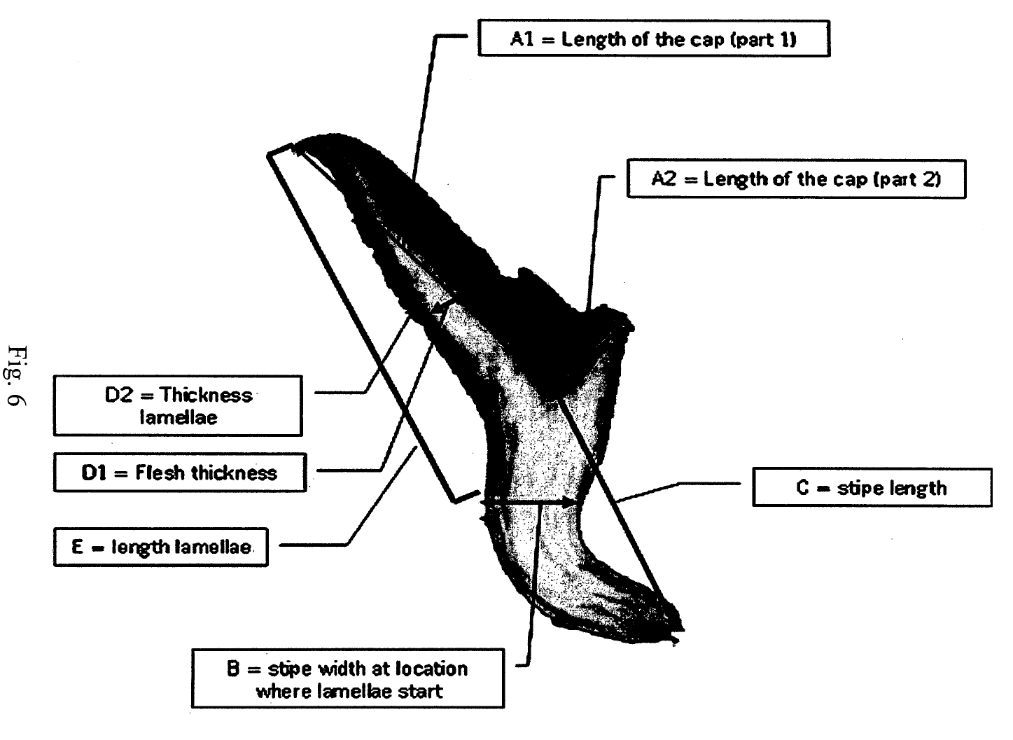

### DETAILED DESCRIPTION OF THE INVENTION

The following detailed description sets forth the breeding procedures and the characteristics of the new cultivar. As shown in Schematic 1, in breeding the new variety, parental lines were isolated from HK35 (female parent) (unpatented) and ATCC 58937 (male parent) (unpatented). Each of the parental lines of the former strain was mated with a different parental line of the latter strain. Offspring was used to find linkage of genetic marker to the “sporeless” trait. The trait was subsequently introgressed in both parental lines of HK35 by repeated backcrosses. Finally, both recombinant parental lines were intercrossed resulting in a sporeless commercial strain. The vegetative mycelium is maintained both on agar slants and in a vapour phase of liquid nitrogen.

The new variety is similar in shell-shaped morphology, mushroom yield and well-developed flesh to HK35, but it produces no spores like ATCC 58937. ‘Spoppo’ has sporeless basidia like ‘Somycel 3210’ (U.S. Pat. No. 4,658,083), but has shell-shaped, well-developed fleshy mushrooms unlike ‘Somycel 3210’. Further, unlike ‘Somycel 3210’, the new variety has no axial symmetry and no positive geotropism. Characteristics that distinguish ‘Spoppo’ from other varieties known to the breeder include:
Basidia produce no spores
Well developed fleshy and heavy caps
Shell-shaped mushrooms 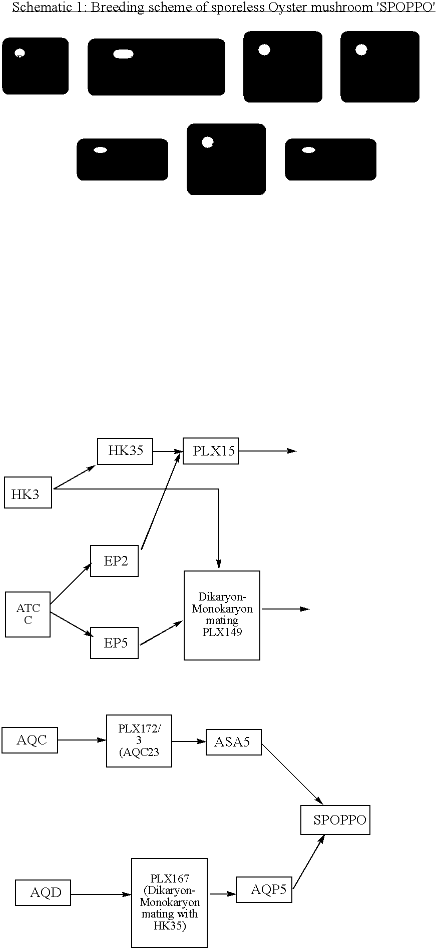
Botanical data collection conditions and standards:

The color standard used in the present application is the L*a*b* method (also designated as CIE.LAB or CIE-L*a*b*) defined by “Commission International de l'Eclaire” in 1976. The location where measurements were made was Horst-America, The Netherlands. Light conditions where measurements were made were by artificial light provided by a Minolta Chroma Meter type CR-200.
Growth conditions:

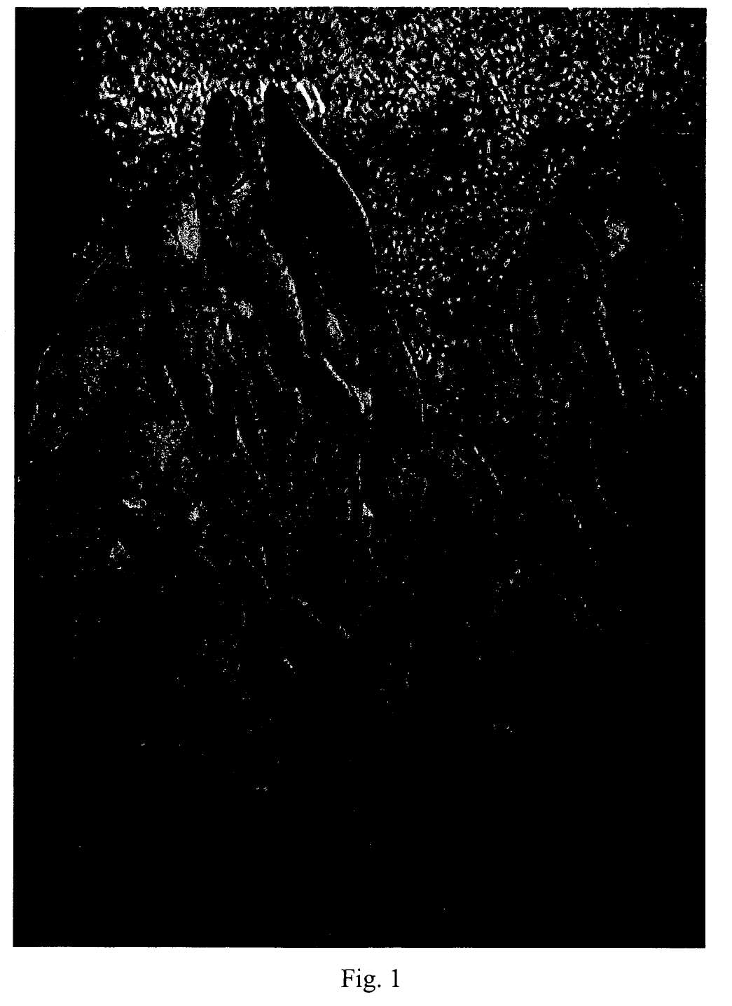

The oyster mushrooms were grown on fermented wheat straw that is packed into 17-18 kg blocks that are wrapped in micro-porous plastic foil. Before packaging, the wheat straw substrate was inoculated with spawn at 25-30 liters per 1000 kg of substrate. The yield is variable, as with all mushroom growing. The yield varied between 150 kg and 250 kg per 1000 kg of substrate.
Botanical description:
Age of described plant: Oyster mushrooms are generally produced within 5 weeks after inoculation of the straw substrate. The Oyster mushrooms are produced as a bundle of mushrooms varying in size. This limits the possibilities for a uniform description of the mushrooms.
Cap:
   Shape.—Shell formed. Stipe is attached excentric.
   Diameter.—Range of 4-15 cm.
   Color.—L: 64.11 (st. dev. 7.55), a: 3.59 (st. dev. 0.87), b: 13.10 (st. dev. 1.81) (see FIG. 1).
   Flesh color.—The color of the mushroom flesh was measured on a longitudinal cut mushroom, using a Minolta Chromameter according to the CIELAB system. L value varies between 80.29 and 92.50 (average 87.35, st. dev. 3.55, n=26) “a” value varies between 0.14 and 1.23 (average 0.80, st. dev. 0.30, n=26) “b” value varies between 3.59 and 10.46 (average 6.38, st. dev. 1.51, n=26).
   Color designation of bruising/cutting.—Change of color after cutting the mushroom was measured on the same mushrooms as used for measuring the “Flesh Color” above. designation”. Cut Oyster mushrooms were incubated in a humid chamber for 2 hours at room temperature Subsequently color of the flesh was determined using a Minolta Chromameter according to the CIELAB system. L value varies between 78.70 and 92.99 (average 88.12, st. dev. 3.35, n=26) “a” value varies between 0.08 and 1.54 (average 0.83, st. dev. 0.27, n=26) “b” value varies between 3.72 and 8.12 (average 5.84, st. dev. 1.07, n=26) There was only a very small change in color compared to the color of freshly cut Oyster mushroom.
   Texture.—No scales present, the cap is smooth.
Gills:
   Characteristics.—The lamellae of strain ‘SPOPPO’ radiate from the stipe outward to the margin of the cap. Intermediate lamellae start at some point on the cap and radiate from that point outward to the margin of the cap. The length of the lamellae varies between 22 and 165 mm (average 69.3 mm, st. dev. 25.0 mm, n=159). The thickness of the lamellae varies between 1 and 12 mm (average 7.0 mm, st. dev. 1.6, n=159). On a cross section of the cap, on average, 13.7 lamellae are placed adjacent per cm (st. dev. 1.3, n=20).
   Color designation.—The color of the lamellae was measured using a Minolta Chromameter according to the CIELAB system. L varies between 73.75 and 81.42 (average 78.51, st. dev. 2.05, n=21) “a” value varies between 0.00 and 0.92 (average 0.25, st. dev. 0.25, n=21) “b” value varies between 7.92 and 12.43 (average 9.37, st. dev. 1.24, n=21).
Stipe:
   Placement.—The mushrooms are uplifted infundibuliform with an excentric attached stipe.
   Shape.—The stalk is bent around the bottom and small. It becomes broader near the cap.
   Size.—The length of the stipe varies from 10 to 110 mm (average 54.8 mm, standard deviation 17.9 mm, number of observations 159). The thickness of the stipe varies from 7 to 28 mm (average 15.5 mm, standard deviation 4.5 mm, number of observations 159).
   Pubescence.—None present.
Spores: None present.
Veil: No veil present.
General:
Disease resistance: Not determined.
Shipping quality: Spawn can be stored (for up to 3 months) and shipped at 4° C. Harvested mushrooms can be stored at 4° C. for less than two weeks.
Temperature tolerance: Between 0 and 30° C.

I claim:

1. A new and distinct variety of Pleurotus ostreatus mushroom as described and illustrated.
